(19) United States
(12) Patent Application Publication
(10) Pub. No.: US 2003/0212037 A1
(43) Pub. Date: Nov. 13, 2003

(54) Use of bvdu for inhibiting the growth of hyperproliferative cells

(76) Inventors: Christopher Boyer, San Diego, CA (US); David B. Lackey, San Diego, CA (US)

Correspondence Address: Antoinette F Konski, McCutchen Doyle Brown & Enersen, 18th Floor, Three Embarcadero Center, San Francisco, CA, 94111-4067, US

(21) Appl. No.: 10/168,722

(22) PCT Filed: Dec. 21, 2000
(86) PCT No.: PCT/US00/35027

(51) Int. Cl. A61K 31/7072; A61K 31/56

(52) U.S. Cl. 514/50; 514/171

(57) ABSTRACT

This invention provides methods for selectively killing a hyperproliferative cell by contacting the cell with the compound BVdU, its derivatives and pharmaceutically acceptable salts. Further provided by this invention is a method for treating a pathology in a subject characterized by pathological, hyperproliferative cells by administering to the subject an effective amount of the compound BVdU, its derivatives and pharmaceutically acceptable salts. The invention also provides a method for screening for potential therapeutic agents by contacting a neoplastic cell with the agent and with BVdU and performing an assay to detect inhibition of proliferation and cell killing. The invention also provides methods for selecting from among a patient population, patients that are likely to benefit from treatment with BVdU, by determining the level of endogenous, intracellular TK and TS. The invention also provides methods for sensitizing patients to the therapeutic effects of BVdU by treatment with substances that result in the increase in the levels of TK in hyperproliferative cells.

### CROSS-REFERENCE TO RELATED APPLICATIONS

The present application claims priority under 35 U.S.C. §119 (e) to U.S. Provisional Application Serial Nos. 60/171,971 and 60/173,996, filed Dec. 23, 1999 and Dec. 30, 1999, respectively, the contents of which are hereby incorporated by reference into the present disclosure.

### TECHNICAL FIELD

The present invention relates to the field of drug discovery and specifically, to methods of using the compound BVdU, BVdU derivatives and pharmaceutically acceptable salts of these compounds to inhibit the growth of hyperproliferative cells.

### BACKGROUND

Uncontrolled growth, de-differentiation and genetic instability characterize Cancer cells. The instability expresses itself as aberrant chromosome number, chromosome deletions, rearrangements, loss or duplication beyond the normal dipoid number (Wilson, J. D. et al., 1991). This genomic instability may be caused by several factors. One of the best characterized is the enhanced genomic plasticity which occurs upon loss of tumor suppression gene function (e.g., Almasan, A. et al. 1995). The genomic plasticity lends itself to adaptability of tumor cells to their changing environment, and may allow for the more frequent mutation, amplification of genes, and the formation of extrachromosomal elements (Smith, K. A. et al., 1995 and Wilson, J. D. et al., 1991). These characteristics provide for mechanisms resulting in more aggressive malignancy because it allows the tumors to rapidly develop resistance to natural host defense mechanisms, biologic therapies (Wilson, J. D. et al., 1991 and Shepard, H. M. et al., 1988), as well as to chemotherapeutics (Almasan, A. et al., 1995 and Wilson, J. D. et al., 1991).

Cancer is one of the most commonly fatal human diseases worldwide. Treatment with anticancer drugs is an option of steadily increasing importance, especially for systemic malignancies or for metastatic cancers that have passed the state of surgical curability. Unfortunately, the subset of human cancer types that are amenable to curative treatment today is still rather small (Haskell, C. M. eds. 1995, p. 32). Progress in the development of drugs that can cure human cancer is slow. The heterogeneity of malignant tumors with respect to their genetics, biology and biochemistry as well as primary or treatment-induced resistance to therapy mitigate against curative treatment. Moreover, many anticancer drugs display only a low degree of selectivity, causing often severe or even life threatening toxic side effects, thus preventing the application of doses high enough to kill all cancer cells. Searching for anti-neoplastic agents with improved selectivity to treatment-resistant pathological, malignant cells remains therefore a central task for drug development.

Accordingly, there is a need for more selective agents that can penetrate the tumor and inhibit the proliferation and/or kill cancer cells. The present invention satisfies this need and provides related advantages as well.

### DISCLOSURE OF THE INVENTION

Methods for inhibiting the proliferation of a hyperproliferative cell are provided by this invention. The methods require contacting the cell with an effective amount of (E)-5-(2-bromovinyl)-2′-deoxyuridine (also called bromovinyl deoxyuridine, BVdU) a derivative of BVdU or a pharmaceutically acceptable salt thereof. The hyperproliferative cells overexpress the enzyme thymidylate synthase (TS) or thyrnidine kinase (TK) as compared to normal, healthy cells. The contacting can be in vitro or in vivo. When performed in vitro, the method provides a means to determine when a cell, tumor or tissue will be responsive to BVdU therapy. In vivo, the method provides a therapy to inhibit or stop the growth or proliferation of cells susceptible to BVdU therapy, e.g., cells resistant to the anti-cancer drugs producing TS overexpression, e.g., Tomudex, N10-propargyl-58-dideazafolic acid (CB3717) and N⁶-[4-(morpholinosulfonyl)benzyl]-N⁶-methyl-2,6-diaminobenz-[c,d]-indole glucuronate (“AG331”).

The invention also provides a method for screening for potential therapeutic agents by separately contacting samples of neoplastic cells with the agent and with BVdU and performing an assay to detect inhibition of proliferation of cell growth.

Additionally, the invention provides a method for identifying individual cancer patients from among a patient population that are most likely to benefit from the administration of BVdU, by assaying biopsy or other tissue samples for thymidine kinase (TK) and thymidylate synthase (TS) enzyme levels.

### MODES FOR CARRYING OUT THE INVENTION

Throughout this disclosure, first author and date, patent number or publication number reference various publications. The full bibliographic citation for each reference can be found at the end of this application, immediately preceding the claims. The disclosures of these references are hereby incorporated by reference into this disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. These methods are described in the following publications.

Definitions

As used herein, certain terms may have the following defined meanings.

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “subject” or “host” is a vertebrate, preferably an animal or mammal, more preferably a human patient. Mammals include, but are not limited to, murines, simians, human patients, farm animals, sport animals, and pets.

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

As used herein, “inhibit” means to delay or slow the growth, proliferation or cell division of cells.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 15th Ed. (Mack Publ. Co., Easton (1975)).

An “effective amount” is an amount sufficient to effect beneficial or desired results. For example, a therapeutic amount achieves the desired therapeutic effect. This amount may be the same or different from a prophylatically effective amount that will prevent onset of disease or disease symptoms. An effective amount can be administered in one or more administrations, applications or dosages.

(E)-5-(2-bromovinyl)-2′-deoxyuridine (also called bromovinyl deoxyuridine, BVdU and BVDU) can be prepared by methods that are well-known in the art. For example, treatment of 5-chloromercuri-2′-deoxyuridine with haloalkyl compounds, 5 haloacetates or haloalkenes in the presence of Li₂PdCl₄ results in the formation, through an organopalladium intermediate, of the 5-alkyl, 5-acetyl or 5-alkene derivative, respectively (Wataya, et al., 1979 and Bergstrom, et al, 1981).

Alternatively, BVdU and its monophosphate derivative are available commercially from Glen Research, Sterling, Va. (USA), Sigma-Aldrich Corporation, St. Louis, Mo. (USA), Moravek Biochemicals, Inc., Brea, Calif. (USA), ICN, Costa Mesa, Calif. (USA) and New England Nuclear, Boston, Mass. (USA). Commercially available BVdU can be converted to its monophosphate either chemically or enzymatically, through the action of a kinase enzyme using commercial available reagents from Glen Research, Sterling, Va. (USA) and ICN, Costa Mesa, Calif. (USA).

Salts of the BVdU may be derived from inorganic or organic acids and bases. Examples of acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, can be employed in the preparation of salts useful as intermediates in obtaining salts of BVdU. Examples of bases include alkali metal (e.g., sodium) hydroxides, alkaline earth metal (e.g., magnesium) hydroxides, ammonia, and compounds of formula NW₄⁺, wherein W is C₁₋₄ alkyl.

Examples of salts include: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylproprionate, picrate, pivalate, propionate, succinate, tartrate, thiocyanate, tosylate and undecanoate. Other examples of salts include anions of the compounds of the present invention compounded with a suitable cation such as Na⁺, NH₄⁺, and NW₄⁺ (wherein W is a C₁₋₄ alkyl group).

Derivatives of BVdU include esters. Esters of BVdU include carboxylic acid esters (i.e., —O—C(═O)R) obtained by esterification of the 2′-, 3′- and/or 5′-hydroxy groups, in which R is selected from (I) straight or branched chain alkyl (for example, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkylsulfonyl (for example, methanesulfonyl) or aralkylsulfonyl; (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di-(C₆₋₂₄)acyl glycerol. In such esters, unless otherwise specified, any alkyl moiety present advantageously-contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms.

Ethers of BVdU include methyl, ethyl, propyl, butyl, isobutyl, and sec-butyl ethers.

The present invention provides methods for inhibiting the growth or viability of a hyperproliferative cell that endogenously overexpresses an intracellular enzyme by contacting the cell with an effective amount of BVdU, a derivative or a pharmaceutically acceptable salt thereof. As used herein, the term “a hyperproliferative cell” is intended to encompass cells dividing at an increased rate above what is considered to be the normal level. In most cases hyperproliferation is due to genetic mutation or endogenous overexpression of cellular enzymes controlling the rate of cell division. Applicants have discovered that hyperproliferative cells such as neoplastic cells overexpressing TS or TK are particularly sensitive or responsive to the anti-proliferative effects of BVdU. Indeed, one can determine which hyperproliferative cells, and therefore patients, that are most responsive to BVdU therapy by assaying a sample of the cells obtained by biopsy or otherwise for the TS or TK expression level. Cells expressing high levels of these either proteins (at least 3× and more preferably at least 4×) have been shown to be particularly sensitive to the anti-proliferative effects of BVdU. In one aspect, TS overexpression is the result of prior treatment with a drug such as Tomudex, N10-propargyl-58-dideazafolic acid (CB3717) and N⁶-[4-(morpholinosulfonyl)benzyl]-N⁶-methyl-2,6-diaminobenz-[c,d]-indole glucuronate (“AG331”). In an alternative aspect, TK overexpression is the result of prior treatment with an estrogen, e.g., estradiol, estradiol valerate, estradiol cyprionate, estradiol decanoate, estradiol acetate, and ethinyl estradiol. Another aspect of this invention is reversing resistance to drug resistance, wherein the drug resistance is the result of overexpression of an endogenous, intracellular enzyme by contacting the cell with an effective amount of BVdU. Examples of such drugs include, but are not limited to Tomudex, N10-propargyl-58-dideazafolic acid, N⁶-[4-(morpholinosulfonyl)benzyl]-N⁶-methyl-2,6-diaminobenz-[c,d]-indole glucuronate, or an estrogen, for example, estradiol, estradiol valerate, estradiol cyprionate, estradiol decanoate, estradiol acetate, or ethinyl estradiol.

Neoplastic cells that are preferentially responsive to BVdU therapy include cells that are de-differentiated, immortalized, neoplastic, malignant, metastatic or transformed. Neoplastic or cancer cells include, but are not limited to a sarcoma cell, a leukemia cell, a carcinoma cell, or an adenocarcinoma cell. More specifically, the cell can be a breast cancer cell, a hepatoma cell, a colorectal cancer cell, pancreatic carcinoma cell, an oesophageal carcinoma cell, a bladder cancer cell, an ovarian cancer cell, a skin cancer cell, a liver carcinoma cell, or a gastric cancer cell. In another aspect of the invention, the hyperproliferative cell is a cell characterized as having an inactivated tumor suppressor function, e.g., loss or inactivation of retinoblastoma (RB) or p53, tumor suppressor genes known to be mutated in a significant fraction of human tumor cells.

The contacting can be in vitro or in vivo and when used herein, contacting is intended to include in vitro or in vivo without expression. When the method is practiced in vitro, it provides a means to determine the efficacy of BVdU therapy on a particular cell type or for a particular patient by contacting a biopsy sample with BVdU. Therapeutic in vivo administration is used to inhibit, stop or reduce the growth of hyperproliferative cells or tumors or to relieve the symptoms associated with presence of hyperproliferative cells, e.g., cachexia. In vivo administration is used to treat pathologies associated with the presence of hyperproliferative cells or tumors. These pathologies include, but are not limited to pre-malignant growth of tumors, malignant and metastatic tumor growth. Therapeutic amounts can be empirically determined and will vary with the pathology being treated, the subject being treated and the toxicity of the compound. BVdU is particularly useful to treat patients that have developed resistance to other chemotherapeutics, as described above. Moreover, after treatment with BVdU, resistance to the primary drug is reversed and the primary drug can be therapeutically administered once more.

When delivered to an animal, the method is useful to further confirm BVdU as an efficacious therapy or a new candidate agent. As an example of an animal model, groups of nude mice (Balb/c NCR nu/nu female, Simonsen, Gilroy, Calif.) are each subcutaneously inoculated with about 10⁵ to about 10⁹ hyperproliferative, cancer or target cells as defined herein. When the tumor is established, the BVdU, a derivative or salt thereof, is administered, for example, by subcutaneous injection around the tumor. Tumor measurements to determine reduction of tumor size are made in two dimensions using venier calipers twice a week. Other animal models may also be employed as appropriate (Lovejoy, et al., 1997 and Clarke, R., 1996).

Administration in vivo can be effected in one dose, continuously or intermittently throughout the course of treatment. Methods of determining the most effective means and dosage of administration are well known to those of skill in the art and will vary with the compound used for therapy, the purpose of the therapy, the cell and patient being treated. Single or multiple administrations can be carried out with the dose level and pattern being selected by the treating physician. BVdU, derivatives and pharmaceutically acceptable salts thereof can be used in the manufacture of medicaments and for the treatment of humans and other animals by administration in accordance with conventional procedures, such as an active ingredient in pharmaceutical compositions.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to a compound of the present invention, the pharmaceutical compositions can also contain other pharmaceutically active compounds or a plurality of compounds of the invention.

More particularly, a compound of the formula of the present invention also referred to herein as the active ingredient, may be administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the preferred route will vary with the condition and age of the recipient, and the disease being treated.

In general, a suitable dose for each of the above-named compounds, is in the range of about 1 to about 100 mg per kilogram body weight of the recipient per day, preferably in the range of about 1 to about 50 mg per kilogram body weight per day and most preferably in the range of about 1 to about 25 mg per kilogram body weight per day. Unless otherwise indicated, all weights of active ingredient are calculated as the parent compound of the formula of the present invention for salts or esters thereof, the weights would be increased proportionately. The desired dose is preferably presented as two, three, four, five, six or more sub-doses administered at appropriate intervals throughout the day. These sub-doses may be administered in unit dosage forms, for example, containing about 1 to about 100 mg, preferably about 1 to above about 25 mg, and most preferably about 5 to above about 25 mg of active ingredient per unit dosage form. It will be appreciated that appropriate dosages of the compounds and compositions of the invention may depend on the type and severity and stage of the disease and can vary from patient to patient. Determining the optimal dosage will generally involve the balancing of the level of therapeutic benefit against any risk or deleterious side effects of the treatments of the present invention.

Ideally, the compounds of the invention should be administered to achieve peak concentrations of BVdU at sites of disease. This may be achieved, for example, by the intravenous injection of BVdU, optionally in saline, or orally administered, for example, as a tablet, capsule or syrup containing the active ingredient. Desirable blood levels of the compound may be maintained by a continuous infusion to provide a therapeutic amount of BVdU within disease tissue. The use of operative combinations is contemplated to provide therapeutic combinations requiring a lower total dosage of BVdU or another active compound than may be required when each individual therapeutic compound or drug is used alone, thereby reducing adverse effects.

While it is possible for BVdU to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier, which constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. BVdU can also be presented a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and optionally one or more excipients or diluents.

For diseases of the eye or other external tissues, e.g., mouth and skin, the formulations are preferably applied as a topical ointment or cream containing the active ingredient in an amount of, for example, about 0.075 to about 20% w/w, preferably about 0.2 to about 25% w/w and most preferably about 0.5 to about 10% w/w. When formulated in an ointment, the compound may be employed with either a paraffinic or a water-miscible ointment base. Alternatively, the composition ingredients may be formulated in a cream with an oil-in-water cream base.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound, which enhances absorption or penetration of the composition ingredient through the skin or other affected areas. Examples of such dermal penetration enhancers include dimethylsulfoxide and related analogues.

The oily phase of the emulsions of this invention may be constituted from known ingredients in a known manner. While this phase may comprise merely an emulsifier (otherwise known as an emulgent), it desirably comprises a mixture of at lease one emulsifier with a fat or oil or with both fat and oil. Preferably, a hydrophilic emulsifier is included together with a lipophilic emulsifier, which acts as a stabilizer. It is also preferred to include both an oil and fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the composition ingredient. The composition ingredient is preferably present in such formulation in a concentration of about 0.5 to about 20%, advantageously about 0.5 to about 10% particularly about 1.5% w/w.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the composition ingredient, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the composition ingredient.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein recited, or an appropriate fraction thereof, of a composition ingredient.

It should be understood that in addition to the ingredients particularly mentioned above, formulations of this invention may include other agents conventional in the art having regard to the type of formulation in question, for example, those suitable of oral administration may include such further agents as sweeteners, thickeners and flavoring agents.

BVdU, its derivatives and salts of BVdU may also be presented for the use in the form of veterinary formulations, which may be prepared, for example, by methods that are conventional in the art.

The invention also provides a method for screening for new potential therapeutic agents by separately contacting samples of hyperproliferative cells with the agent and with BVDU and then performing an assay to detect inhibition of cell proliferation. BVDU is the positive control against which the efficacy of the therapeutic agent is compared. In a further embodiment, a “control” normal, non-neoplastic cell sample is contacted with the test agent and with BVdU. Preferred therapeutic agents will inhibit the growth or viability of hyperproliferative cells but have no effect on the normal, healthy control cells.

Without wishing to be bound to any particular theory, Applicants note that the subject invention relies on a pathway that is distinct from methods and compositions disclosed in PCT Publication No. WO 99/23104. This publication discloses uridine analogs that are activated by replacement of the 5-sbustituent of the base. The authors theorized that following entry into the cell and phosphorylation, an analogue of dUrd serves as a prodrug if TS can methylate it to generate the corresponding dThd analogue.

In contrast, BVdU is not activated by this mechanism and therefore the compounds and methods disclosed in WO 99/23104 are not predictive of the selective therapeutic efficacy of BVdU. Applicants believe that BVdU cytotoxicity to hyperproliferative cells involves conversion to BVDUMP by TK or other enzymes. BVdU is not activated by replacement of the bromovinyl group with a methyl group. In fact, if replacement of the bromovinyl group with a methyl group by TS were to occur, the result would be the natural product dTMP, which is not cytotoxic.

Cell samples can be obtained from biopsies or transformed cells that overexpress the intracellular enzyme. Examples of cell lines useful for such cell assays are ras-transformed NIH 3T3 cells (obtained from the ATCC) and human colorectal and breast tumor cell lines. Alternatively, animal models are useful to test for new therapeutics.

An assay is then performed to detect any inhibition of proliferation and cell killing by the BVdU and the candidate agent. Cell proliferation and killing are measured by any of a variety of assays that quantitate DNA synthesis or determine the number of viable cells in a sample. For example DNA synthesis can be measured by quantitating incorporation of tritiated thymidine or other labeled deoxynucleotide into DNA. Alternatively, the number of viable cells can be measured by various methods such as by using a redox indicator like alamarBlue to quantitate cellular metabolism or by directly counting viable cell. A positive outcome occurs when an agent inhibits the proliferation or kills a neoplastic cell but has a significantly reduced affect on a normal cell when applied at the same concentration. A significantly reduced affect occurs when the test agent preferentially kills neoplastic cells with about 2-fold and preferably about 3-fold or greater activity than normal cells.

A prognostic test is further provided by this invention. The expression level or amount of TS or TK is measured using methods described herein or well known to those of skill in the art. Cells that overexpress TS or TK at least 3× or more preferably 4× as compared to normal cells of the same type are beneficially treated by BVdU therapy.

In addition, one can enhance the therapeutic benefit of BVDU by prior administration of an agent or drug known to enhance TK expression, examples of which are provided herein. In addition, this invention provides a method to ameliorate the carcinogenic effect of estrogen and other drugs known to enhance TK expression and thus hyperproliferation by co-administration or subsequent administration of BVdU.

The following examples are intended to illustrate, but not limit the inventions described herein.

### EXPERIMENTAL EXAMPLES

Cell strains and Cell Lines. Cell lines used in experiments described below are SW527P (normal breast tissue), SKBR3 V (breast adenocarcinoma cell line stably transfected with control vector only), CCD18co (normal colon cell strain), Det551 (normal colon cell strain, MCF7 (breast cancer cell line). SKBR3 #52 (breast adenocarcinoma transfected with thymidylate synthase expression vector), HT1080 #12 (fibrosarcoma cell line stably transfected with thymidylate synthase expression vector), SW527 TDX, H630-R10, and MCF7 TDX.

alamarBlue cytotoxicity assay Tumor cells growing exponentially were transferred to 384 well flat bottom tissue culture plates. H630 RIO were plated at a density of 500 cells per well and MCF7 TDX at 250 cells/well in 25 μL of complete medium (RPMI 1640+10% fetal bovine serum+antibiotics/antimyotics). After 24 hours (day 0), 25 μL of complete medium containing the compounds (NB1011 or BVDU) over the dose range of 10⁻³ to 10⁻¹⁰ M were added in triplicate. Drug exposure time was 120 hours (day 5), after which growth inhibition was assayed. The redox indicator alamarblue was added to each well at 10% (v/v). After 1-hour incubation at room temperature, fluorescence was monitored at 536 nm excitation and 595 nm emission. Concentration versus relative fluorescence units (RFU) was plotted, and sigmoid curves were fit using the Hill equation. IC₅₀, indicated by the inflection point of the curve, is the concentration at which growth is inhibited by 50%.

Cyquant and Crystal Violet Cytotoxicitv Exponentially growing cells were transferred at a density of 1.0-4.0×10³ cells per well to a 96-well tissue culture plate in growth medium (RPMI 1640+10% FBS+antibiotics). Cells were allowed to attach for 24 hours in standard culture conditions (37° C., 5% CO₂, 95% humidity). Experimental compounds were then applied in duplicate half log serial dilutions. After additional 72 hour incubation, surviving cells were stained with crystal violet (adherent cells) or Cyquant (semi or non-adherent cells). Absorbence or fluorescence, respectively, was monitored. IC₅₀ values were derived from sigmoid curves fit according to the Hill inhibitory Emax model.

Construction of TS mammalian expression vector. The 5′ base pairs of TS cDNA was modified by decreasing the GC content without changing the amino acids they encoded and additional DNA fragment was introduced to encode a 6 histidines tagged to N-terminal of TS. The cDNA was subcloned into XhoI and HindIII sites of mammalian expression vector pcDNA3. I (−). The cDNA insert was confirmed by DNA sequencing.

Cell transfection. HT1080 cells were grown in RPMI1640 medium supplemented with 10% FBS, and transfected with TS expression vector. 48 hours later, transfected cells were trypsinized and replated in culture medium containing 750 μg/ml G418. After selection with G418 for two weeks, surviving cells were cloned. Clones with different TS levels were selected based on Western blot analysis, and expanded into cell lines. The stable HT1080 cells transfected with pcDNA3.1(−) only were used as control.

Antitumor Cell Efficacy of BVdU on the Breast Cancer Cell Line MCF7 TDX The efficacy of BVdU in inhibiting the proliferation of a test cancer cell line was demonstrated by comparison with the deoxyribose nucleotide derivative NB1011 using a cell-based assay. NB1011 {(E)-5-(2-bromovinyl)-2′deoxyuridine phenyl L-alaninylphosphoramidate)} is a modified derivative of BVdUMP with a neutral 5′-phosphoramidates, L-phenyl L-alaninlyphosphoramidate. The process for preparing NB 1011 is known in the art (See PCT/US99/01332).

H630 R10 is a colon cancer tumor cell line selected for resistance to 5-FU, and overexpresses thymidylate synthase protein approximately 20-fold. MCF7 TDX is a breast tumor cell line selected with Tomudex, and overexpresses thymidylate synthase to approximately the same extent. Both cell lines are sensitive to NB1011 compared to normal cell strains; however, MCF7 TDX is significantly more sensitive to NB1011 than is H630 R10. H630 R10 has previously been shown to be insensitive to BVdU.

The efficacy of BVdU in inhibiting the proliferation of a selected tumor cell line was demonstrated by determining the IC₅₀ using the alamarBlue cytotoxicity assay described above.

TABLE 1

| Compound | H630 R10 IC₅₀ (μM) | MCF7 TDX IC₅₀ (μM) |
|---|---|---|
| NB1011 | 57 | 0.13 |
| BVdU | 303 | 0.005 |

These results indicate that BVdU is relatively inactive against H630R10 cells (fluoropyrimidine resistant colon) (303 μM IC₅₀, ˜6 fold less active than NB1011). In contrast, it was found that BVdU was extremely cytotoxic against MCF7 TDX cells (Tomudex resistant breast cancer cell line), (5 nM IC₅₀, ˜25-fold more active than NB1011. This finding shows that a class of tumor cells exists with sensitivity to BVdU, similar to that of MCF7 TDX cells, and that tumor cells of this type are potential targets for BVdU therapy.

Further experiments indicate that a range of tumor cell types, including breast and colon tumors, are sensitive to the anti-proliferative effects of BVdU, whereas normal cell strains representing colon and skin are not affected by even high concentrations of BVdU. The tumor cell types tested include tumor cell lines resistant to 5-FU and Tomudex, drugs that are clinically accepted as cancer therapy.

TABLE 2

| | | TS Protein | | BvdU | 5-FU | BVdU |
|---|---|---|---|---|---|---|
| Cell | | Level | 5-FU | IC₅₀ | normal | normal |
| Designation | Description | (Units) | IC₅₀ (μM) | (μM) | tumor | tumor |
| SW527P | tumor-breast, | 22 | 9.1 ± 1.3 | >1000 | | |
| | parental control | | | | | |
| SKBR3 V | tumor-breast, | 64 | 7.4 ± 2.4 | >1000 | | |
| | vector control, | | | | | |
| | low TS | | | | | |
| CCD18co | normal colon | 100 | 1.4 ± 0.4 | 4822 ± 128 | | |
| | epithelium | | | | | |
| Det 551 | normal embryonic | 177 | 3.1 ± 0.5 | 2194 ± 682 | | |
| | skin | | | | | |
| MCF7 | tumor-breast, | 178 | 8.8 ± 5.9 | 1251 | 0.25 | 2.8 |
| | parental control | | | | | |
| SKBR3 #52 | tumor-breast | 590 | 9.1 ± 1.6 | 8.3 | 0.24 | 423 |
| | high TS | | | | | |
| | transfectant | | | | | |
| TS HT1080 | tumor- | 678 | 3.5 ± 0.2 | 5.7 ± 1.8 | 0.64 | 615 |
| #12 | fibrosarcoma, high | | | | | |
| | TS transfectant | | | | | |
| SW527TDX | tumor-breast, | 980 | 20.4 ± 9.9 | 6.5 | 0.11 | 540 |
| | TDX resistant, | | | | | |
| | high TS | | | | | |
| H630-R10 | tumor-colon, 5- | 2405 | 143 ± 5.9 | 561 ± 157 | 0.15 | 6.3 |
| | FU resistant, high | | | | | |
| | TS | | | | | |
| MCF7TDX | tumor-breast, | 2581 | 6.7 ± 2.0 | 0.6 ± 0.5 | 0.33 | 5847 |
| | TDX resistant, | | | | | |
| | high TS | | | | | |

The selectivity of a given antitumor agent can be assessed by comparing the IC₅₀ for a tumor cell line to the IC₅₀ of a normal cell strain growing under the same conditions, determined and Cyquant staining for non-adherent cells, and crystal violet staining for adherent cells. The selectivity is given here as the ratio of normal cell IC₅₀ to tumor cell IC₅₀. In this case, normal cell IC₅₀ is defined as the average of the value for CCD18co and Det551 to allow for direct comparison of the established cancer drug 5-FU with BVdU.

The results of this experiment indicate that BVdU is more than ten times as selective as 5-FU when tested on the breast cancer cell line MCF7. Tumor cell lines that express elevated levels of thymidylate synthase are in general much more sensitive to BVdU. For example, the tumor cell line SKBR3 #52 has a normal/tumor ratio of 423 for BVdU (that is, 2,000 times higher than the normal/tumor ratio of 5-FU). Similar results were obtained for TS HT1080 #12 normal/tumor ratio 615 (961 times the 5-FU normal/tumor ratio), and SW527TDX 540 (5,000 times the 5-FU normal/tumor ratio), and MCF7 TDX 5847 (18,000 times the 5-FU normal/tumor ratio). The exception to this rule was H630-R10, which had a normal/tumor ratio of 6.3 (42 times the normal/tumor ratio of 5-FU). The H630 R-10 cell line is unique in that it has been selected for 5-FU resistance (and higher TS activity) by passage in media containing 5-FU. Similarly, the MCF7 TDX tumor cell line has an exceptionally high normal/tumor ratio of 5847 (5847 (18,000 times the 5-FU normal/tumor ratio). The high TS level in the MCF7 TDX tumor cell line is the result of selection for Tomudex resistance by passage in media containing Tomudex.

These results provide a means for identifying tumor types that may be especially susceptible to the antitumor effects of BVdU. As discussed above, tumor cell lines that over express TS are generally quite sensitive to BVdU, and have normal/tumor IC₅₀ ratios much better than 5-FU, indicating potential clinical benefit. Predictably, the cell line with the highest TS level (MCF7 TDX) also has the lowest IC₅₀ for BVdU, and the highest normal/tumor ratio. Therefore, one can predict that tumors that have become resistant to the cancer drug Tomudex, and which have a high level of TS, are most sensitive to BVdU. In contrast, when the high tumor TS level is the result of selection by 5-FU treatment, as with H630-R10 cells, BVdU is predicted to be much less effective as an anticancer agent than with the other TS over-expressing tumor cell lines. Therefore, these novel and unexpected findings show that BVdU will be exceptionally beneficial against tumors that have acquired resistance to Tomudex or other antifolates due to increased levels of TS.

Cell lines that overexpress human thymidine kinase (TK) are also more sensitive to BVdU, providing another criterion for identification of tumor susceptibility to BVdU. Table 3 shows the results of an experiment in which the HT1080 human fibrosarcoma cell line and stably transfected cell lines expressing elevated levels of human thymidine kinase were compared for sensitivity to BVdU. Transfected cell lines with increasing levels of thymidine kinase demonstrate a progressive increase in sensitivity to BVdU.

TABLE 3

| | TK Protein | BVdU IC50 |
|---|---|---|
| Cell Type | Level (Units) | (μM) |
| PC HT1080 | 100 | 303 |
| TKC HT1080 #5 | 200 | 275 |
| TKC HT1080 #22 | 400 | 162 |

This observation indicates that tumor cells containing elevated levels of thyrnidine kinase or other enzymes that can convert BVdU to BVdUMP will be a diagnostic indicator of tumor cell sensitivity to BVdU.

Selection of patients likely to benefit. It has previously been shown that individual human tumors contain levels of thymidine kinase that vary widely in enzyme activity and isozyme composition of thymidine kinase (Madec, A. et al 1988), and (Stafford, M. A., and Jones, O. W. 1972). The results shown herein enables the selection of patients that will benefit from treatment with BVdU by identifying tumors that express high levels of thymidine kinase or other enzymes that convert BVdU to BVdUMP (TK), as well as thymidylate synthase (TS), simultaneously.

Elevated levels of thymidine kinase can be measured by a number of well known methods, including cytofluorometric methods that provide for the measurement of thymidine phosphorylation in individual cells, whether by thymidine kinase or other enzymes (Hengtschlager, M., and Wawra, E. 1993), (Hengtschlager, M., and Wawra, E., 1993), (Hengtschlager, M., and Bemaschek, G., 1997). Other methods applicable to the quantitation of elevated thymidine kinase levels include immunofluorescence using specific antibody to thymidine kinase, and the use of DNA probes with specific sequences that hybridize with thymidine kinase mRNA, couple with methods for detecting hybridization, such as RT-PCR, and other well-established methods in molecular biology.

Use of thymidine kinase induces to sensitize tumors. In addition to identifying patients whose tumors have intrinsic, pre-existing elevated levels of thymidine kinase, our innovation enables the use of BVdU in tumors containing elevated levels of thymidine kinase resulting from the application of a thymidine kinase inducing agent. Because of our discoveries relating to the enhanced effectiveness of BVdU as an anticancer agent in cells containing elevated levels of thymidine kinase, it will be possible to sensitize tumors to BVdU by the application of agents that cause elevated levels of thymidine kinase. An example of one such agent is estradiol, which is known to induce elevated levels of thymidine kinase in human breast cancer tumors (Bronzert, D. A., et al 1981).

In vivo Testing

Ras-transformed NIH 3T3 cell lines are transplanted subcutaneously into immunodeficient mice. Initial therapy may be direct intratumoral injection. Inhibition of tumor growth is measured by comparing the rate of increase in tumor size in comparison with control samples receiving a carrier composition without active agent. Similar studies may be performed with human tumors derived from various stages of disease progression, from multiple individuals or from alternative tissue types. Optionally, experiments are performed as above except the drug will be administered intravenously into the animals to address issues related to efficacy, toxicity and pharmacobiology of the drug candidates. The in vivo studies will be conducted as described by Harris, M P et al. (1996) and Antelman, D. et al. (1995).

While the invention has been described in detail herein and with reference to specific embodiments thereof, it will be apparent to one skilled in the art that various changes and modifications can be made to the invention as described above without departing from the spirit and scope thereof.

### REFERENCES

Literature

Almasan, A. et al. (1995) Cancer Metastases Rev. 14:59-73

Antelman, D. et al. (1995) Oncogene 10:697

Barr, P. J. et al. (1983) J. Biol. Chem. 258(22):13627-31

Bergstrom, et al. (1981) J. Org. Chem. 46:1432-1441

Bigge, et al (1980) J. Amer. Chem. Soc. 102:2033-2038

Bronzert, D. A., et al (1981) Cancer Research 41, 604-610.

Clarke, R. (1996) Brest Cancer Res. Treat. 39:1-6

Dale, et al. (1973) Proc. Natl. Acad. Sci. USA 70:2238-2242

Dorr, R. T. and Von Hoff, D. D., eds. “Cancer Chemotherapy Handbook” 2nd ed. (Appleton and Lange 1994), pp. 768-773, 1020

Harris, M. P. et al. (1996) Cancer Gene Therapy 3:121

Haskell, C. M. ed. Cancer Treatment 4th Ed., J. Dyson, Ed., (Philadelphia: W. B. Saunders Co. 1995)

Hengstschlager, M., and Bemaschek (1997) G. FEBS Lett. 404, 299-302.

Hengstschlager, M., and Wawra, E. (1993) Cytometry 14, 39-45.

Hengstschlager, M., and Wawra, E. (1993) Br. J. Cancer67, 1022-1025

Lovejoy, et al. (1997) J. Pathol. 181:130-5

Madec, A., et al (1988) Bull. Cancer 187-194.

Shepard, H. M. et al. (1988)J. Clin. Immunol. 8:353-395

Smith, K. A. et al. (1995) Philos Tran Royal Soc 347:49-56

Stafford, M. A., and Jones, O. W. (1972) Biochimica et Biophysica ACTA 277, 439-442.

Wataya, et al. (1979) J. Med. Chem. 22:339-340

Wilson, J. D., et al. (eds.) “Harrison's Principles of Internal Medicine” (12^{th} ed) (McGraw-Hill, Inc. 1991) 2208, esp. 21-76

Patent Documents

International Patent Application No. PCT/US99/01332 for “Enzyme Catalyzed Therapeutic Agents”

International Patent Publication No. WO 99/23104 for “Nucleosides for Imaging and Treatments Applications

U.S. Pat. No. 4,247,544, Bergstrom, D. E. et al. “C-5 Substituted Uracil Nucleosides”, issued Jan. 27, 1981

U.S. Pat. No. 4,267,171, Bergstrom, D. E. et al. “C-5 Substituted Cytosine Nucleosides” issued May 12, 1981

U.S. Pat. No. 4,948,882, Ruth, J. L. “Single-Stranded Labelled Oligonucleotides, Reactive Monomers and Methods of Synthesis” issued Aug. 14, 1990

What is claimed is:

1. A method for selectively inhibiting the proliferation of a hyperproliferative cell endogenously overexpressing an intracellular enzyme, comprising contacting the cell with an effective amount of (E)-5-(2-bromovinyl)-2′deoxyuridine, a derivative or a pharmaceutically acceptable salt thereof.

2. The method of claim 1, wherein the hyperproliferative cell is characterized as having an inactivated tumor suppressor function.

3. The method of claim 1, wherein the hyperproliferative cell is a neoplastic cell.

4. The method of claim 1, wherein the intracellular enzyme is thymidylate synthase.

5. The method of claim 1, wherein the intracellular enzyme is thymidine kinase.

6. The method of claim 4, wherein overexpression of thymidylate synthase is the result of prior chemotherapy.

7. The method of claim 5, wherein overexpression of thymidine kinase is the result of prior chemotherapy.

8. The method of claim 7, wherein the chemotherapy is an estrogen is selected from the group consisting of estradiol, estradiol valerate, estradiol cyprionate, estradiol decanoate, estradiol acetate, and ethinyl estradiol.

9. The method of claim 3, wherein the neoplastic cell is selected from the group consisting of a breast cancer cell, a hepatoma cell, a colorectal cancer cell, pancreatic carcinoma cell, an oesophageal carcinoma cell, a bladder cancer cell, an ovarian cancer cell, a skin cancer cell, a liver carcinoma cell, or a gastric cancer cell.

10. A method for treating a pathology in a subject characterized by hyperproliferative cells endogenously overexpressing an intracellular enzyme in a subject comprising administering to the subject an effective amount of (E)-5-(2-bromovinyl)-2′deoxyuridine, a derivative or a pharmaceutically acceptable salt thereof.

11. The method of claim 10, wherein the hyperproliferative cell is characterized as having an inactivated tumor suppressor function.

12. The method of claim 10, wherein the hyperproliferative cell is a neoplastic cell.

13. The method of claim 12, wherein the intracellular enzyme is thymidylate synthase.

14. The method of claim 13, wherein the intracellular enzyme is thymidine kinase.

15. The method of claim 13, wherein overexpression of thymidylate synthase is the result of prior chemotherapy.

16. The method of claim 14, wherein overexpression of thymidine kinase is the result of prior chemotherapy.

17. The method of claim 16, wherein the chemotherapy is an estrogen is selected from the group consisting of estradiol, estradiol valerate, estradiol cyprionate, estradiol decanoate, estradiol acetate, and ethinyl estradiol.

18. The method of claim 12, wherein the neoplastic cell is selected from the group consisting of a breast cancer cell, a hepatoma cell, a colorectal cancer cell, pancreatic carcinoma cell, an oesophageal carcinoma cell, a bladder cancer cell, an ovarian cancer cell, a skin cancer cell, a liver carcinoma cell, or a gastric cancer cell.

19. A method for screening for potential therapeutic agents comprising separately contacting samples of hyperproliferative cells overexpressing an activating intracellular enzyme with a test agent and BVdU, and assaying to detect inhibition of cell proliferation or cell killing of the cell samples.

20. The method of claim 19, further comprising separately contacting a sample of control cells with the test agent and BVdU.

21. The method of claim 20, wherein the control sample is a normal, non-neoplastic, cell type and the hyperproliferative cells are neoplastic cells.

22. A method for reversing resistance in a cell endogenously overexpressing an endogenous, intracellular enzyme as a result of prior chemotherapy comprising contacting the cell with an effective amount of BVdU, a derivative or pharmaceutically acceptable salt thereof.

23. The method of claim 22, wherein the endogenous intracellular enzyme is thymidylate synthase or thymidine kinase.

24. The method of. Claim 22, wherein the prior chemotherapy is selected from the group consisting of Tomudex, N10-propargyl-58-dideazafolic acid, N⁶-[4-(morpholinosulfonyl)benzyl]-N⁶-methyl-2,6-diaminobenz-[c,d]-indole glucuronate, estrogen, estradiol, estradiol valerate, estradiol cyprionate, estradiol decanoate, estradiol acetate, and ethinyl estradiol.

25. A method for determining whether a hyperproliferative cell is suitably treated with BVdU, a derivative or pharmaceutically acceptable salt thereof comprising:
a) determining the endogenous, intracellular expression level of an activating enzyme produced by the hyperproliferative cell;
b) determining the endogenous, intracellular expression level of the enzyme in a corresponding, normal cell;
c) comparing the result of a) and b) to determine if a) is at least 3× higher than b), then the cell is suitably treated.

26. The method of claim 25, wherein the enzyme is intracellular thymidylate synthase or thymidine kinase.

27. A method for minimizing the hyperproliferative effects of an estrogen, comprising administering an effective amount of BVdU, a derivative or pharmaceutically acceptable salt thereof.

28. A method for sensitizing a hyperproliferative cell to the therapeutic effects of BVdU by contacting the cell with an effective amount of an agent that increases the intracellular level of thymidine kinase.

29. The method of claim 27 or 28, wherein the agent is an estrogen.

30. The method of claim 29, wherein the estrogen is selected from the group consisting of estradiol, estradiol valerate, estradiol cyprionate, estradiol decanoate, estradiol acetate, and ethinyl estradiol.
